(19) United States
(12) Patent Application Publication
(10) Pub. No.: US 2024/0035064 A1
(43) Pub. Date: Feb. 1, 2024

(54) METHODS AND DEVICES FOR EVALUATING TEST FORMULATIONS TO DETERMINE BIOLOGICAL AGENT LOG REDUCTION/REMOVAL ON A SURFACE

(71) Applicants: GOJO Industries, Inc., Akron, OH (US); North Carolina State University, Raleigh, NC (US)

(72) Inventors: Clyde S. Manuel, Northfield, OH (US); Taylor Niehaus, Akron, OH (US); Jeremy Faircloth, Raleigh, NC (US); Lee-Ann Jaykus, Durham, NC (US)

(21) Appl. No.: 18/363,046

(22) Filed: Aug. 1, 2023

Related U.S. Application Data
(60) Provisional application No. 63/393,998, filed on Aug. 1, 2022.

(51) Int. Cl. C12Q 1/18

(52) U.S. Cl. CPC C12Q 1/18

(57) ABSTRACT

Exemplary methodologies for testing pathogen reduction/removal, devices for testing pathogen reduction/removal and coupon trays for testing pathogen reduction/removal are disclosed herein. An exemplary methodology for testing pathogen reduction/removal includes providing a pathogen test device that has a base and a wiping block. The wiping block is configured to move back and forth in a linear motion with respect to the base. The test device further includes a tray, a coupon recess located in the tray and an access recess located in the tray. The exemplary methodology further includes securing a wiping substrate to the wiping block, providing a coupon that has a pathogen on a surface of the coupon, placing the coupon in the coupon recess, applying a test product to the coupon, allowing the test product to remain on the coupon for a set time period, and causing the wiping block to move back-and-forth across the surface of the coupon. The methodology further includes applying a selected downward force to the surface of the coupon, inserting at least a portion of a coupon removal tool in the access recess and removing the coupon. The methodology further includes placing the coupon in a neutralizer and determining a log reduction and/or removal of the pathogen from the surface of the coupon.

### RELATED APPLICATIONS

This application claims priority to and the benefits of U.S. Provisional Patent Application Ser. No. 63/393,998, titled “METHODS AND DEVICES FOR EVALUATING TEST FORMULATIONS TO DETERMINE BIOLOGICAL AGENT LOG REDUCTION/REMOVAL ON A SURFACE,” filed on Aug. 1, 2022, and which is incorporated herein in its entirety.

### BACKGROUND OF THE INVENTION

Biological agents, including bacteria and viruses, cause serious illnesses throughout the world. Norovirus is likely the most common cause of gastrointestinal illness in the world and is sometimes called a “perfect pathogen”. The virus spreads very easily and, if not properly controlled, could lead to an epidemic. According to the Centers of Disease Control and Prevention (CDC), norovirus is responsible for approximately 21 million cases of acute gastroenteritis per year, and norovirus accounts for more than 50 percent of food-borne diseases in America. Norovirus infection occurs via the fecal oral route, and the virus is highly stable in the environment, surviving for weeks on hard surfaces. Contamination of surfaces serves as a significant reservoir and transmission vector for norovirus. In fact, contaminated surfaces often lead to cross contamination with food, which can cause outbreaks. The most common settings for foodborne transmission of norovirus are food service venues, so wherever food is being prepared, there is the possibility of a norovirus contamination event and the potential for an outbreak. Recent CDC statistics demonstrate that restaurants are responsible for 64% of norovirus outbreaks.

Unlike most bacteria, norovirus cannot be easily cultured in a growth medium which makes it a difficult organism to study. Cultivation of norovirus is difficult and requires a tissue culture resembling cells in the organism it infects, in this case, humans. While a cell culture model to propagate norovirus was reported in 2016, it does not function with all norovirus strains, is expensive, time consuming, and requires highly skilled labor, and cannot be used routinely. The source of norovirus for laboratory studies comes from the fecal material of infected persons, and because this material is limited, only very tiny quantities of norovirus are used in laboratory testing of an antimicrobial product's ability to inactivate and/or remove norovirus from a surface. Heretofore, product testing has been limited to small quantities of norovirus on small stainless-steel or glass coupons. This is not realistic when compared to in-field use on large surfaces such as tables and countertops.

A prior art testing system, known as “Quantitative Assessment of Microbial Decontamination and Transfer Using a Carrier Platform” (“QTC3”) has been used for such testing. The QTC3 device uses small round stainless-steel coupons that are loaded in a tray. When the end of the product test time has been reached, the floor of the tray is pulled out and the small round stainless-steel coupons fall into a buffer solution. The coupons are removed from the buffer solution and analyzed for the presence and concentration of virus.

In addition, most restaurants and bars have surfaces that have different properties than stainless steel, such as, for example, laminate counter tops and tabletops. Testing products for their ability to reduce and/or remove norovirus on stainless steel coupons may not be indicative of their ability to reduce and/or remove norovirus from everyday surfaces, such as for example, laminate countertops.

Furthermore, existing test methods that include a wiping step during disinfection of coupons utilize manual techniques after a test product has been applied for a set period of time. Manual wiping techniques are problematic because the amount of force applied during the wiping process is inconsistent, which leads to inconsistent and non-repeatable results. Accordingly, there is a need for methods and systems for testing a product's ability to reduce and/or remove norovirus, or other pathogens, from surfaces, and in particular, from surfaces representative of real-life applications.

### SUMMARY

Exemplary methodologies for testing pathogen reduction/removal, devices for testing pathogen reduction/removal and coupon trays for testing pathogen reduction/removal are disclosed herein. An exemplary methodology for testing pathogen reduction/removal includes providing a pathogen test device that has a base and a wiping block. The wiping block is configured to move back and forth in a linear motion with respect to the base. The test device further includes a tray, a coupon recess located in the tray and an access recess located in the tray. The exemplary methodology further includes securing a wiping substrate to the wiping block, providing a coupon that has a pathogen on a surface of the coupon, placing the coupon in the coupon recess, applying a test product to the coupon, allowing the test product to remain on the coupon for a set time period, and causing the wiping block to move back-and-forth across the surface of the coupon. The methodology further includes applying a selected downward force to the surface of the coupon, inserting at least a portion of a coupon removal tool in the access recess and removing the coupon. The methodology further includes placing the coupon in a neutralizer and determining a log reduction and/or removal of the pathogen from the surface of the coupon.

An exemplary pathogen test device includes a base, a track, a trolley, a support arm connected to the trolley, a wiping block, the wiping block connected to the support arm with a pivot member, and a tray. The tray has one or more coupon recesses for holding a coupon and one or more access recesses. The one or more access recesses intersect with the one or more coupon recesses. Wherein the trolley travels in a back-and-forth linear motion along the track and the wiping block travels back and forth over the one or more coupon recesses. A selected downward force is applied to the wiping block.

An exemplary tray for a pathogen test device includes a base, one or more coupon recesses for holding a test coupon that has a surface with a pathogen thereon. The one or more recesses are in a first surface of the base. The one or more coupon recesses have a depth that is the same as the thickness of a test coupon. The tray further includes one or more access recesses that intersect with the one or more coupon recesses. The one or more access recesses are configured to allow a coupon removal tool to engage the coupon to remove a coupon from the one or more coupon recesses.

### BRIEF DESCRIPTION OF THE DRAWINGS

These and other features and advantages of the present invention will become better understood with regard to the following description and accompanying drawings in which:

FIG. 1 is an exemplary embodiment of a portion of a test device for evaluating test formulations to determine pathogen and/or biological agent log reduction/removal from a surface;

FIG. 2 is an exemplary embodiment of components of a wiping block for the test device of FIG. 1;

FIG. 3 is a partial assembly of the wiping block components of FIG. 2;

FIG. 4 is an assembled wiping block with the components of FIG. 2;

FIG. 5 is a bottom view of the assembled wiping block;

FIG. 6 illustrates the wiping block being attached to the portion of the device of FIG. 1;

FIG. 7 illustrates the wiping block attached to the portion of the device of FIG. 1;

FIG. 8 is an enlarged portion of an exemplary embodiment of a tray for the test device of FIG. 1;

FIG. 9 illustrates a coupon being placed in the tray;

FIG. 10 is an exemplary embodiment of the test device with a coupon in a coupon recces;

FIG. 11 illustrates a coupon removal tool being inserted into an access recess for removal of the coupon from the tray;

FIG. 12 is an exemplary embodiment of a tray for a test device;

FIG. 13 is a cross-section of the tray of FIG. 12;

FIG. 14 is another exemplary embodiment of a tray for a test device;

FIG. 15 is a cross-section of the tray of FIG. 14;

FIG. 16 is another exemplary embodiment of test device; and

FIG. 17 is an exemplary methodology of testing a product formulation for determining the log reduction and/or removal of the biological agent from a surface.

### DETAILED DESCRIPTION

The Detailed Description merely describes exemplary embodiments of the invention and is not intended to limit the scope of the claims in any way. Indeed, the invention is broader than and unlimited by the exemplary embodiments, and the terms used in the claims have their full ordinary meaning. Unless otherwise specified, the term “log reduction” as used herein refers to log₁₀ reduction.

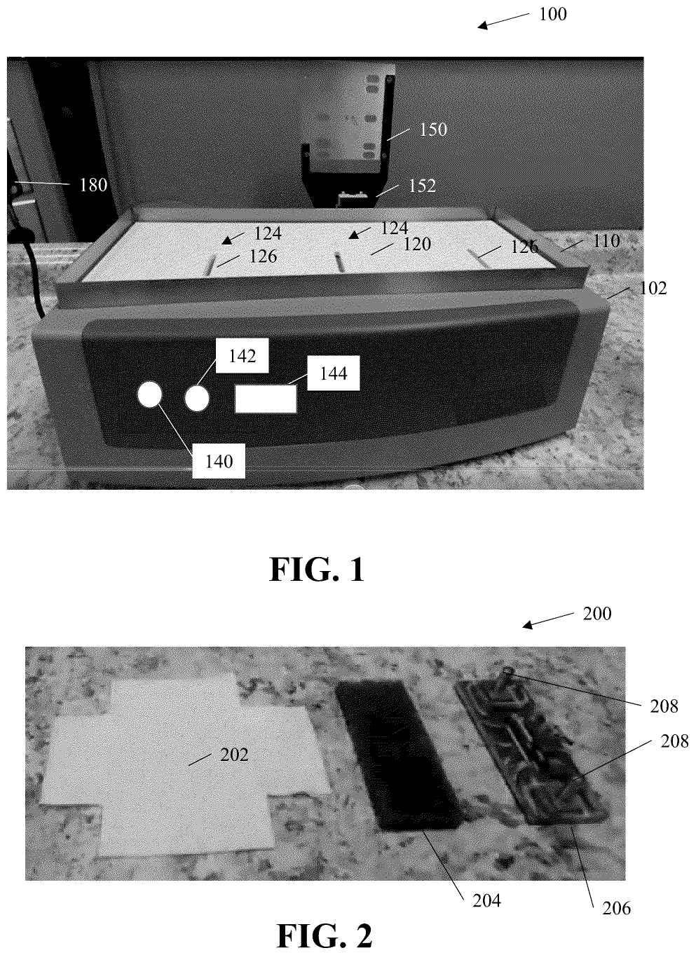

FIG. 1 is an exemplary embodiment of a portion of a test device 100 for evaluating product formulations to determine biological agent/pathogen log reduction/removal from a surface. Test device 100 includes a housing 102. Located within hosing 102, is a motor (not shown), a drive train and/or gearing (not shown) for moving a trolley (not shown) back and forth in a linear motion. In addition, test device 100 includes electronic circuitry (not shown) for controlling operation of the trolley. In this exemplary embodiment, test device 100 includes a plug 180 for providing AC power to power the electronic circuitry. In some embodiments, test device 100 includes one or more batteries (not shown) to provide power to the test device 100.

In addition, test device 100 includes one or more inputs, 140, 142, and an optional display 144. The one or more inputs 140, 142 may be used to, for example, start/stop movement of the trolley, adjust the speed of the trolley, stet the number of times the trolley moves back-and-forth, or the like. In this exemplary embodiment, test device 100 includes software for setting the number of sweeps across the surface of the tray 120.

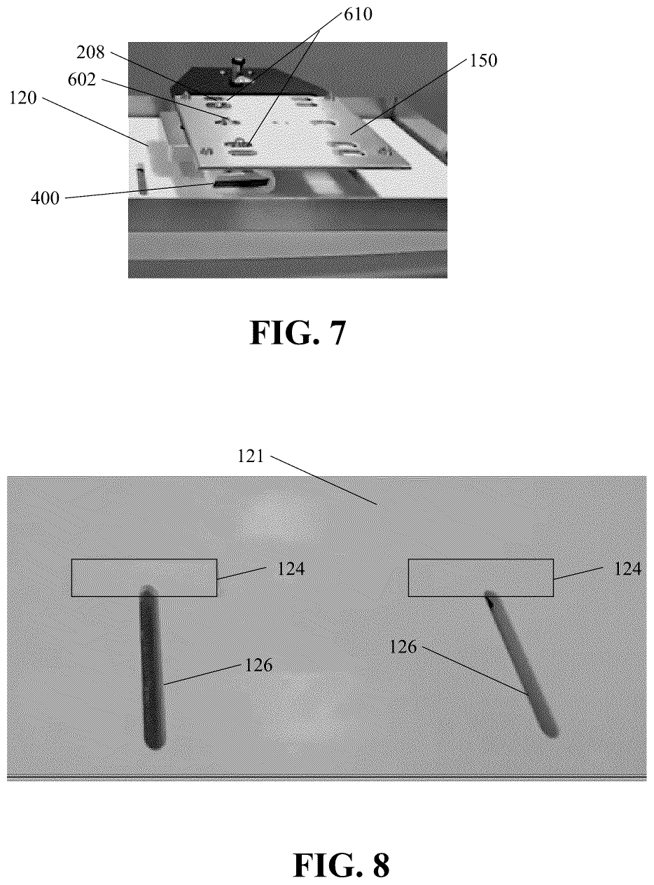

Secured to the trolley (not shown) is a is a swing arm 150. Swing arm 150 is connected to the trolly (not shown) by a hinge 152. Accordingly, in this exemplary embodiment, swing arm 150 may be moved from an upright position as shown in FIG. 1 to a downward position as shown in FIG. 7.

Test device 100 includes a base 110 for receiving a tray 120. In this exemplary embodiment, tray 120 is made out of polypropylene. Other types of material may be used provided they have properties that make them compatible with the product formulation being tested. Preferably, tray 120 is made of a material that is machinable with, for example, a CNC machine. Tray 120 includes one or more coupon recesses 124 and one or more access recesses 126. (See also FIG. 8, which is an enlarged partial view of tray 120). In this exemplary embodiment, coupon recesses 124 have a rectangular shape. In some embodiments, the one or more coupon recesses have a circular shape. In some embodiments, the one or more coupon recesses have rounded edges. In some embodiments, the one or more coupon recesses have one or more tapered edges.

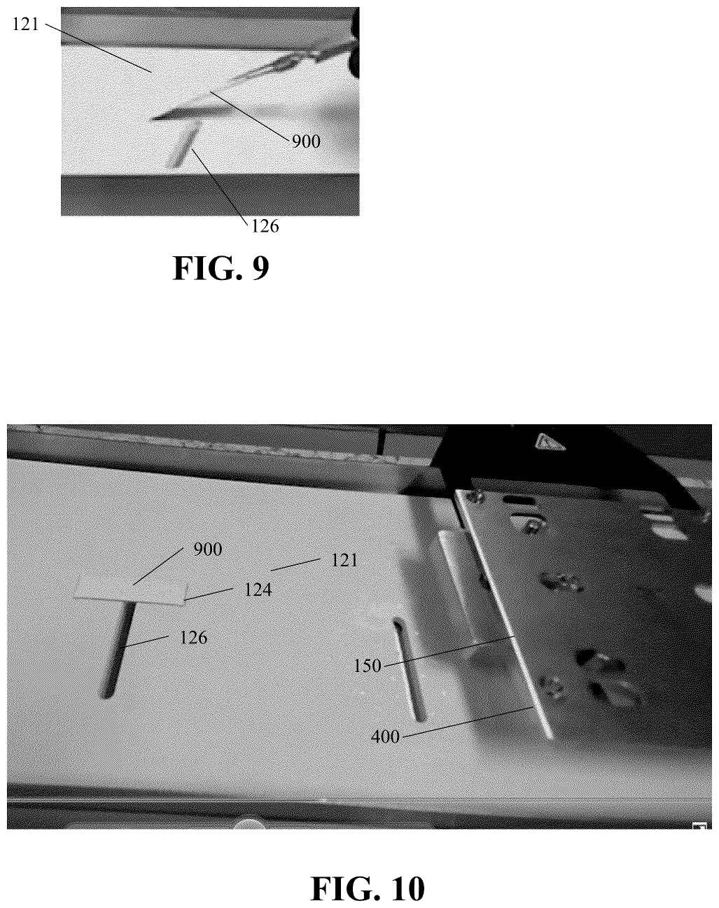

In addition, coupon recesses 124 are configured to have a depth that matches the thickness of a coupon 900 (FIGS. 9, 10). Tray 120 also includes one or more access recesses 126.

In some embodiments, tray 120 and the coupons are made of the same material. In some embodiments, the tray 120 has a textured surface. In some embodiments, the tray 120 and coupon have the same textured surfaces.

FIG. 2 is an exemplary embodiment of components of a wiping block 200. In this exemplary embodiment, wiping block 200 is made up of three components, a wiping substrate 202, a substrate backing member 204 and an attachment block 206. Substrate 202 may be any absorbent material. In some embodiments, substrate 202 is a paper towel. In some embodiments, substrate 202 is a cloth, such as, for example, a microfiber cloth. In some embodiments, substrate 202 is a non-woven material. In some embodiments, substrate 202 is a wipe. In some embodiments, test device 100 and substrate 202 are used to test wet wipes and wipes formulation. Substrate 202 may be other types of material that may be used to wash or clean a surface, such as, for example, a sponge, an abrasive pad, or the like.

In some embodiments, the test device 100 is used to test the effectiveness of the test product. In some embodiments, test device 100 is used to test the effectiveness of the substrate 202 for removal of pathogens. In some embodiments, the test device 100 is used to test the effectiveness of the test product and the substrate 202 in combination.

In this exemplary embodiment, substrate backing member 204 is a resilient member, such as, for example, a foam, a rubber, an extrusion, or the like. In some embodiments, a resilient member serves to ensure that an even force is applied across the entire contact area of the substrate 202. In some embodiments, substrate backing member is a semi-rigid material. In some embodiments, wipes backing member 204 is resilient and has one or more ridges. The one or more ridges may be resilient and have a compression property that simulates a human hand. In some embodiment, wipes backing member 204 has four ridges. In some embodiments, which may be used on a scaled up version, wipes backing member 204 may be resilient and have a raised hand shaped surface to simulate a person wiping a surface.

Attachment block 206 includes one or more block retainer(s) 208. The one or more block retainers 208 are configured to attach wiping block 200 to swing arm 150. The one or more retainers 208 are sized to fit within one or more apertures 610 in swing arm 150. In this exemplary embodiment, the one or more block retainers 208 are cylindrical pegs. In some embodiments, the one or more block retainers 208 are threaded and a nut (not shown) is used to secure the wiping block 200 to the swing arm 150.

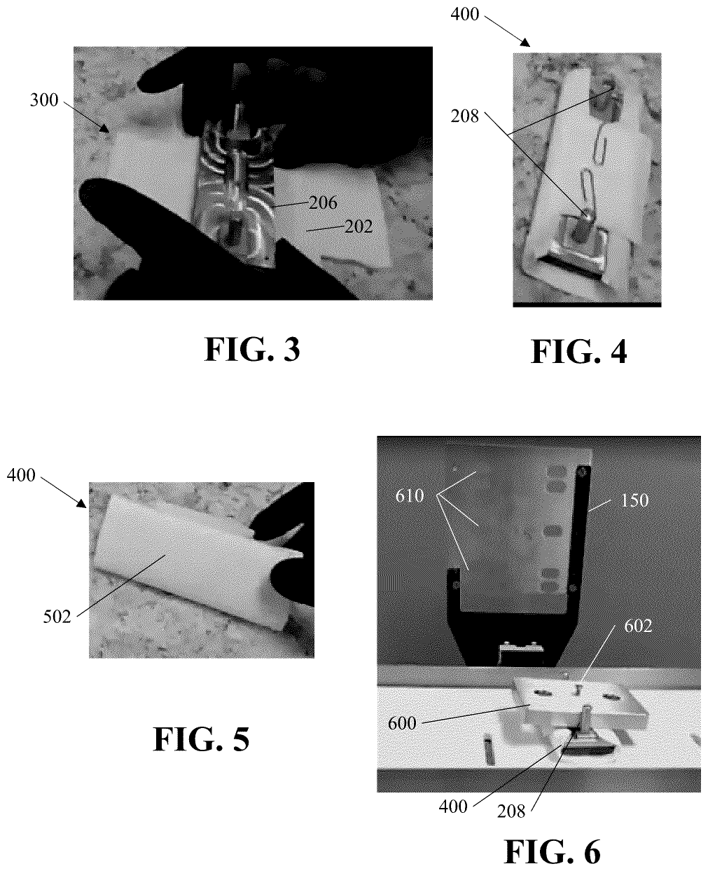

FIG. 3 is a partial assembly of the wiping block 200 with the attachment block 206 and backing member 204 stacked on substrate 202. FIG. 4 shows wiping block 200 in an assembled position, and FIG. 5 is a bottom view of the assembled wiping block 200.

FIG. 6 is a partial view of test device 100 with the wiping block 200 on the tray 120. A weight 600 is placed on top of wiping block 200. Weight 600 includes one or more weight retainers 602. The one or more weight retainers 602 are configured to weight 600 to swing arm 150. The one or more weight retainers 602 are sized to fit within one or more apertures 610 in swing arm 150. In this exemplary embodiment, the one or more weight retainers 602 are cylindrical pegs. In some embodiments, the one or more weight retainers 602 are threaded and a nut (not shown) is used to secure the weight 600 to the swing arm 150.

In some embodiments, a weight 600 is not needed. In some embodiments, wiping block 200 has a sufficient weight and the extra weigh it not needed.

Preferably, wiping block 200 and optional weight 600 have a total weight of at least 150 grams. In some embodiments, wiping block 200 and optional weight 600 have a total weight of at least 200 grams. In some embodiments, wiping block 200 and optional weight 600 have a total weight of at least 250 grams. In some embodiments, wiping block 200 and optional weight 600 have a total weight of at least 300 grams. In some embodiments, wiping block 200 and optional weight 600 have a total weight of at least 350 grams. In some embodiments, wiping block 200 and optional weight 600 have a total weight of at least 400 grams. In some embodiments, wiping block 200 and optional weight 600 have a total weight of at least 450 grams. In some embodiments, wiping block 200 and optional weight 600 have a total weight of at least 500 grams. In some embodiments, wiping block 200 and optional weight 600 have a total weight of at least 550 grams. In some embodiments, the “optional” weight is included in the mass of the wiping block. In some embodiments, wiping block 200 and optional weight 600 have a total weight of at least 600 grams. In some embodiments, the downward force is between 900 grams and 1800 grams. In some embodiments, the downward force is up to about 8000 grams.

In some embodiments, a load cell (not shown) is included in the test device. The load cell is configured to measure the downward force. The load cell provides a signal to a processor (not shown) and memory (not shown) which control a force actuator (not shown) that applies downward force to the wiping block. The force actuator may include, for example, one or more hydraulic or pneumatic cylinders, a motor, any necessary gearing, a force actuator controller, or the like. In some embodiments, the downward force is a constant force throughout the wiping motion. In some embodiments, the downward force is variable throughout the wiping process. In some embodiments, the downward force is selected to simulate the downward force applied by a person's hands when wiping a surface. In some embodiments, the downward force through the middle section of a wipe stroke is greater than the downward force on one or more end sections of the wiping stroke. In some embodiments, the downward force actuator adjusts the downward force one or more times during the length of wipe stroke.

FIG. 7 is a partial prospective view of test device 100 with the swing arm 150 lowered into its downward position with the one or more retaining members 208 in apertures 610 and the one or more weight retaining members 602 in apertures 610. In this exemplary embodiment, apertures 610 are slots. In some embodiments, the apertures have a circular shape and are sized slightly larger than block retaining members 208 and weight retaining members 208.

FIG. 9 illustrates a coupon 900 being placed in the tray 120. Coupon 900 may be made of one or more of a number of different materials that demonstrate the cleaning, sanitizing, and/or disinfecting properties of a cleaning product formulation on each of the different materials. Preferably, coupon 900 is made of a material that the product formulation being tested will be used on. Exemplary materials may include, for example, a laminate material, such as for example, a Formica Brand laminate, stainless steel, marble, granite, stone, tile, or the like. The ability to test the product on specific surfaces where the product will be used is highly valuable as it provides data that is indicative of the disinfecting or sanitizing ability of the product on real life surfaces rather than on a single surface. Moreover, the data may demonstrate certain cleaning product formulations are suitable, or are superior, for certain materials but are not suitable, or are inferior, for certain materials. Thus, if the primary surface that the product will be used on is laminate, the test device may be used to determine that one product formulation provides superior results over other products.

Coupon 900 has a rectangular shape. In some embodiments, coupon 900 has a different shape, such as, for example, a circular shape, an oval shape, a square shape. In some embodiments, coupon 900 has one or more rounded corners. In some embodiments, coupon 900 has a textured surface. In some embodiments, multiple coupons made of different materials are inserted into two or more coupon recesses 124.

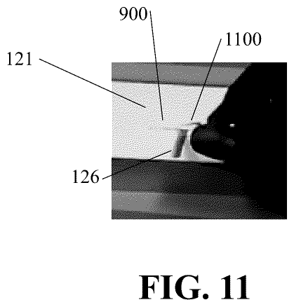

FIG. 10 is a partial view of the test device 100 having coupon 900 inserted into coupon recess 1245. FIG. 11 illustrates a coupon removal tool 1100 being inserted into an access recess 126 for removal of the coupon 900 from the tray 121.

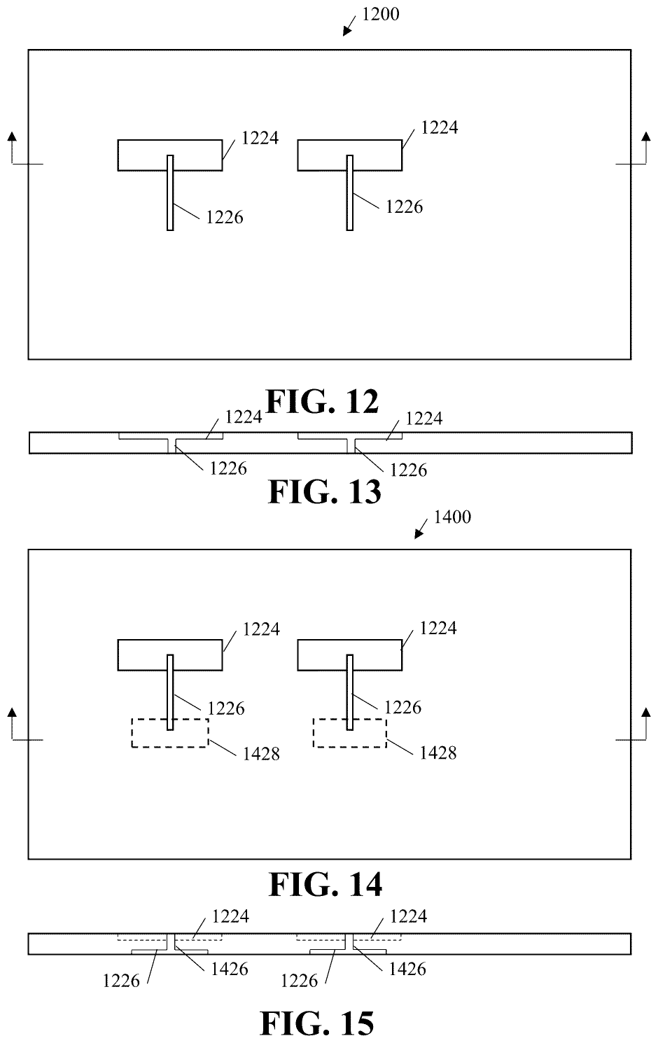

Features disclosed in one embodiment should also be considered as optionally included in other embodiments, without the need to explicitly redescribe each of those features with respect to each embodiment. FIG. 12 illustrates an exemplary embodiment of a tray 1200 for a test device, such as, for example, test device 100. In this exemplary embodiment, tray 1200 is made out of polypropylene. Other types of material may be used provided they have properties that make them compatible with the formulation being tested. Preferably, the materials are machinable, with for example, a CNC machine. As described above, in some embodiments, tray 1200 is made of the same material as the coupon.

Tray 1200 includes one or more coupon recesses 1224 and one or more access recesses 1226. In this exemplary embodiment, coupon recesses 1224 have a rectangular shape. In some embodiments, coupon recesses 1224 have a different shape, such as, for example, a circular shape, an oval shape, a square shape, or the like. In some embodiments, a circular shape, or an oval shape is preferable because there are no corners that may snag the wiping substrate. In some embodiments, rectangular coupon recesses with rounded corners may be preferred. In addition, coupon recesses 1224 have a depth that is configured to match the thickness of a coupon (not shown).

Tray 1200 also includes one or more access recess 1226. Access recess 1226 is configured to allow for quick and easy removal of a coupon from the coupon recess 1224. In this exemplary embodiment, access recess 1226 is a rectangular slot that is sized to allow a coupon removal tool (not shown), such as, for example, tweezers, to be positioned to grip the top and bottom surfaces of a coupon (not shown) for removal of the coupon from the tray 1200. Access recess 1226 may have other shapes, provided the shape allows for easy access for a coupon removal tool. As shown in FIG. 13, in this exemplary embodiment, access recess 1226 extends through tray 1200. In this exemplary embodiment, because at least a portion of access recess 1226 extends through tray 1200, liquid is prevented from pooling in the one or more access recess 1226. In some embodiments, access recess(es) 1226 does not extend through the tray 1200.

FIGS. 14 and 15 illustrate another exemplary embodiment of a tray 1400 for a test device. Tray 1400 is similar to tray 1200, however tray 1400 includes one or more coupon recesses 1428 in a second side of tray 1400. Coupon recesses 1428 may be the same size and depth as the one or more coupon recesses 1224. In some embodiments the one or more coupon recesses 1428 are a different size and/or shape than the one or more coupon recesses 1224. In some embodiments, the one or more coupon recesses 1428 have a different depth than the one or more coupon recesses 1224. In some embodiments, the one or more coupon recesses 1226 are configured to receive coupons made of a different material than the one or more coupon recesses 1224. For example, the one or more coupon recesses 1124 may be configured to receive stainless steel coupons and the one or more coupon recesses 1226 may be configured to receive laminate coupons.

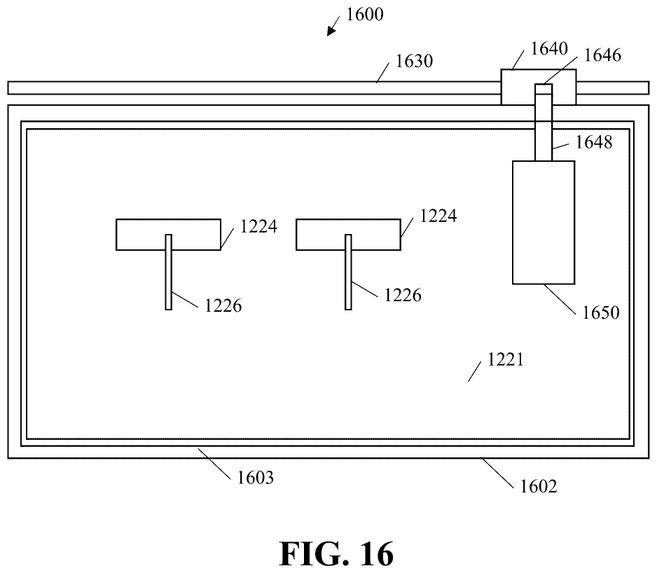

FIG. 16 is another exemplary embodiment of test device 1600 for evaluating product formulations to determine biological agent and/or pathogen log reduction/removal on a surface. The test device 1600 includes a housing 1602. Located within housing 1602, is a motor (not shown), a drive train and/or gearing (not shown) for moving a trolley 1640 back and forth in a linear motion. In addition, test device 1600 includes electronic circuitry (not shown) for controlling operation of the trolley. In this exemplary embodiment, test device 1600 is preferably connectable to AC power to power the electronic circuitry. In some embodiments, test device 1600 includes one or more batteries (not shown) to provide power to the test device 1600.

In addition, test device 1600 includes one or more inputs (not shown) and an optional display (not shown). The one or more inputs may be used to, for example, start/stop movement of the trolley, set the speed of the trolley, stet the number of times the trolley moves back-and-forth, or the like.

Secured to the drive train/gearing 1640 is a swing arm 1648. Swing arm 1648 is connected to the trolly 1640 by a hinge member 1646. Accordingly, in this exemplary embodiment, swing arm 1648 may be moved from an upright position or to a downward position. Connected to swing arm 1648 is a wiping block 1650. Wiping block 1650 is configured to have a wiping substrate (not shown) secured thereto. Wiping block 1650 may include a resilient backing member (not shown).

Trolly 1640 moves in a linear motion along rail 1630. Rail 1630 may be a track, a rack, a guide rail, or the like that causes trolley 1640 to move in a controlled back and forth motion. An optional weight (not shown) may be used to provide a controlled downward force on the substrate (not shown).

Test device 1600 incudes a base 1603 for receiving a tray, such as for example, tray 1200.

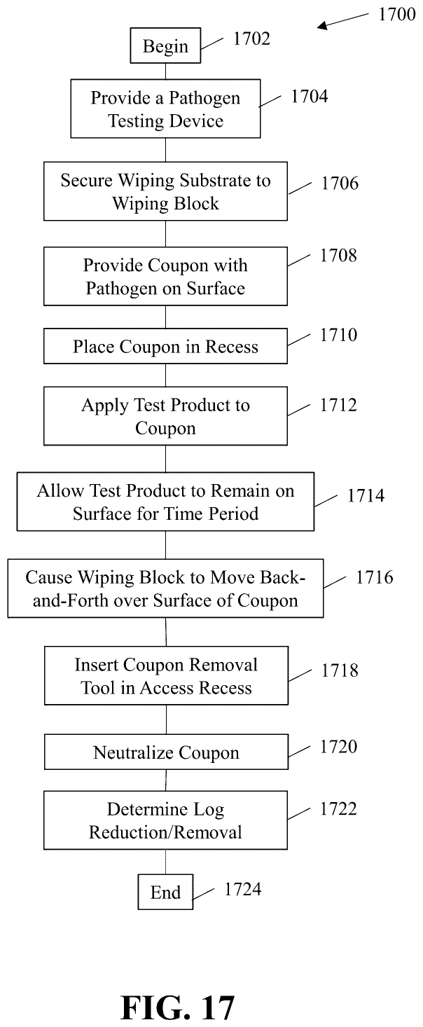

FIG. 17 is an exemplary methodology of testing a product formulation for determining the log reduction and/or removal of the biological agent from a surface. The exemplary methodology is described as having steps or blocks. Additional blocks may be included within the scope of the invention. In addition, blocks may be eliminated within the scope of the invention. Further, although the blocks are shown and described in one order, they could be performed in a different order. The exemplary methodology begins at block 1702. At block 1704 a pathogen test device is provided. The pathogen test device may be one of those shown and described above. At block 1706 a wiping substrate is secured to a wiping block of the pathogen test device. A coupon having a surface inoculated with a pathogen is provided at block 1708. The coupon is placed in a coupon recess at block 1710.

A test product is applied to the surface of the coupon at block 1712 and is allowed to remain on the surface for a specified time period. In some embodiments, the specified time period is 30 seconds. In some embodiments, the specified time period is 60 seconds. In some embodiments, the specified time period is greater than or less than 60 seconds. In some embodiments, the specified time period is between 30 seconds and 60 seconds.

At block 1716, the exemplary methodology the wiping block is caused to move back-and-forth over the surface of the coupon. In some embodiments, the wiping block passes over and back for a total of 3 back-and-forth passes. In some embodiments, the wiping block passes over and back for a total of more than 3 back-and-forth passes. In some embodiments, the wiping block passes over and back for a total of less than 3 back-and-forth passes.

In some embodiments, the time required for the wiping block to pass over and back on the coupon is included in the specified period of time. In other words, if the set period of time is 60 seconds, the wiping of the coupon is set to be completed at the end of the set period of time.

In some embodiments, the set period of time expires prior to the wiping process. In some embodiments, one pass over the coupon with the wiping block may be sufficient, accordingly, the wiping block does not need to pass both over and back over the coupon. In some embodiments, the wiping block passes over the coupon 8 times. In some embodiments, the wiping block passes over the coupon 7 times. In some embodiments, the wiping block passes over the coupon 6 times. In some embodiments, the wiping block passes over the coupon 5 times. In some embodiments, the wiping block passes over the coupon 4 times. In some embodiments, the wiping block passes over the coupon 3 times. In some embodiments, the wiping block passes over the coupon 2 times. In some embodiments, the wiping block passes over the coupon 1 times. In some embodiments, the wiping block passes over the coupon more than 8 times.

The wiping block is configured to apply a constant and repeatable downward force so that the same repeatable downward force can be applied across multiple product tests.

The coupon is removed at block 1718 by insertion of a coupon removal tool in the access recess to remove the coupon. The coupon is place in a neutralizing solution at block 1720. Log reduction by inactivation and/or removal of the pathogen being tested is determined at block 1722 and the methodology ends at block 1724.

In some embodiments, the pathogen is norovirus. In some embodiments, the pathogen is a non-enveloped virus, such as hepatitis A virus, poliovirus, or adenovirus. In some embodiments, the pathogen is an enveloped virus, such as influenza virus or human coronaviruses. In some embodiments, the pathogen is a bacteria, such as Escherichia coli, Listeria monocytogenes, or Salmonella species.

In some embodiments, a second coupon having a pathogen applied to the surface is inserted in a second coupon recess, and the wiping procedure covers the first and second coupons. In this exemplary embodiment, the log reduction/removal determination may be made on both coupons.

In some embodiments, a second coupon that is sterilized is placed in a second coupon recess. The wiping procedure wipes across both the first and second coupons. In this exemplary embodiment, the log concentration determination is made on the second coupon, which is indicative of a pathogen transfer from the first coupon to the second coupon.

While various inventive aspects, concepts and features of the inventions may be described and illustrated herein as embodied in combination in the exemplary embodiments, these various aspects, concepts and features may be used in many alternative embodiments, either individually or in various combinations and sub-combinations thereof. Unless expressly excluded herein all such combinations and sub-combinations are intended to be within the scope of the present inventions. Still further, while various alternative embodiments as to the various aspects, concepts and features of the inventions—such as alternative materials, structures, configurations, methods, circuits, devices and components, software, hardware, control logic, alternatives as to form, fit and function, and so on—may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the present inventions even if such embodiments are not expressly disclosed herein. Additionally, even though some features, concepts or aspects of the inventions may be described herein as being a preferred arrangement or method, such description is not intended to suggest that such feature is required or necessary unless expressly so stated. Still further, exemplary or representative values and ranges may be included to assist in understanding the present disclosure; however, such values and ranges are not to be construed in a limiting sense and are intended to be critical values or ranges only if so expressly stated. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention. Descriptions of exemplary methods or processes are not limited to inclusion of all steps as being required in all cases, nor is the order that the steps are presented to be construed as required or necessary unless expressly so stated.

We claim:

1. A method for testing pathogen reduction/removal comprising:
providing a pathogen test device;
wherein the pathogen test device has
a base;
a wiping block;
wherein the wiping block is configured to move back and forth in a linear motion with respect to the base; and
a tray;
a coupon recess located in the tray;
an access recess located in the tray;
securing a wiping substrate to the wiping block;
providing a coupon that has a pathogen on a surface of the coupon;
placing the coupon in the coupon recess;
applying a test product to the coupon;
allowing the test product to remain on the coupon for a set time period;
causing the wiping block to move back-and-forth across the surface of the coupon;
applying a selected constant downward force to the surface of the coupon during the back- and forth movement of the wiping block;
inserting at least a portion of a coupon removal tool in the access recess and removing the coupon;
placing the coupon in a neutralizer solution; and
determining a log reduction and/or removal of the pathogen from the surface of the coupon.

2. The method of claim 1 further comprising:
a second coupon;
wherein the second coupon has the pathogen applied to a surface of the second coupon;
placing the second coupon in a second coupon recess;
wherein the test product is applied to the surface of the second coupon;
wherein the wiping block is configured to move in a back and forth motion over the second coupon recess;
allowing the test product to remain on the surface of the second coupon for a set period of time;
placing the second coupon in a neutralizing solution;
determining the log reduction and/or removal of pathogen from the surface of the second coupon; and
comparing the log reduction and/or removal of pathogen from the surface of the coupon with the log reduction and/or removal of pathogen from a second coupon.

3. The method of claim 1 further comprising providing a coupon having a surface that is non-metallic.

4. The method of claim 1 further comprising providing a coupon having a surface that is a laminate material.

5. The method of claim 1 further comprising providing one or more weights for applying the selected constant downward force on the wiping block.

6. The method of claim 1 further comprising creating a coupon recess that has a depth that is equal to a thickness of the coupon.

7. The method of claim 1 causing the wiping block to move across the surface of the coupon three or more times.

8. The method of claim 1 the coupon is made of a first material and further comprising a second coupon made of a second material and placing the second coupon in a second coupon recess.

9. The method of claim 1 further comprising placing a second coupon in a second coupon recess, wherein the second coupon is sterilized.

10. The method of claim 9 wherein the wiping block moves back and forth over both the surface of the first coupon and the surface of the second coupon.

11. The method of claim 10 further comprising determining a pathogen transfer on the second coupon.

12. A pathogen test device comprising:
a base;
a track;
a trolley;
a support arm connected to the trolley;
a wiping block;
the wiping block connected to the support arm with a pivot member;
a tray;
the tray having one or more coupon recesses for holding a coupon;
the tray having one or more access recesses, wherein the one or more access recesses intersect with the one or more coupon recesses;
wherein the one or more access recesses are configured to allow a coupon removal tool to engage the coupon to remove a coupon from the one or more coupon recesses;
wherein the trolley travels in a back-and-forth linear motion along the track; and
wherein the wiping block travels back and forth over the one or more coupon recesses; and
wherein a selected constant downward force is applied to a surface of a coupon.

13. The pathogen test device of claim 12 further comprising a weight for providing the selected constant downward force.

14. The pathogen test device of claim 12 further comprising a load cell.

15. The pathogen test device of claim 12 further comprising a force actuator.

16. The pathogen test device of claim 12 wherein the one or more access recesses extend through the tray.

17. The pathogen test device of claim 12 wherein the one or more coupon recesses have a depth that is the same depth as a thickness of the coupon.

18. The pathogen test device of claim 12 wherein the coupon is made of a non-metallic material.

19. The pathogen test device of claim 12 wherein the coupon is a laminate material.

20. A tray for a pathogen test device comprising:
a base;
one or more coupon recesses for holding a test coupon that has a surface with a pathogen thereon;
wherein the one or more recesses are in a first surface of the base
wherein the one or more coupon recesses have a depth that is the same as the thickness of a test coupon;
one or more access recesses;
wherein the one or more access recesses intersect with the one or more coupon recesses;
wherein the one or more access recesses are configured to allow a coupon removal tool to engage the coupon to remove a coupon from the one or more coupon recesses.
